(19) United States
(12) Patent Application Publication
(10) Pub. No.: US 2007/0224590 A1
(43) Pub. Date: Sep. 27, 2007

(54) Single cell planar chromatography

(75) Inventor: Mark G. Knize, Tracy, CA (US)

Correspondence Address: Eddie E. Scott;Assistant Laboratory Counsel, Lawrence Livermore National Laboratory, P.O. Box 808, L-703, Livermore, CA, 94551, US

(73) Assignee: The Regents of the University of California

(21) Appl. No.: 11/387,291

(22) Filed: Mar. 22, 2006

(51) Int. Cl. C12Q 1/00; C12M 3/00; G01N 33/558

(52) U.S. Cl. 435/4; 436/514; 435/287.2

(57) ABSTRACT

A system for preparing a biological cell for analysis comprising the deposition of the biological cell on a planar chromatographic support medium and lysis of the biological cell. In one embodiment, the present invention includes one or two dimensional chromatography of the biological cell. One embodiment of the present invention provides a planar chromatographic support medium including a planar unit and a porous material, a device for deposition of the biological cell on the planar chromatographic support medium, a device for cell lysis of the biological cell, and a chromatography device for one or two dimensional chromatography of the biological cell.

The United States Government has rights in this invention pursuant to Contract No. W-7405-ENG-48 between the United States Department of Energy and the University of California for the operation of Lawrence Livermore National Laboratory.

### BACKGROUND

1. Field of Endeavor

The present invention relates to chromatography and more particularly to single cell planar chromatography.

2. State of Technology

U.S. Pat. No. 6,395,178 issued to Heinz-Emil Hauck May 28, 2002 for thin porous layers for thin-layer chromatography provides the following state of technology information: “Increasing demands made of the performance of analytical methods, in particular in respect of speed and detection sensitivity, mean that in the case of thin layer chromatography (planar chromatography) there is a need for separation medium layers which are significantly thinner than those obtainable hitherto, i.e., thinner than 50 μm.”

U.S. Pat. No. 5,208,458 issued to Kenneth L. Busch and Stephen M. Brown, Jr. issued May 4, 1993 for an interface device to couple gel electrophoresis with mass spectrometry using sample disruption provides the following state of technology information: “An interface for the direct introduction of a sample taken from a planar electrophoresis into a mass spectrometer or other spectrometric device. The interface comprises a probe to collect a sample from a planar electrophoresis, generally a gel electrophoresis, and a filter and guard column configuration to remove unwanted and unnecessary impurities and excess solvent, and to concentrate the sample prior to introduction to the mass spectrometer.”

### SUMMARY

Features and advantages of the present invention will become apparent from the following description. Applicants are providing this description, which includes drawings and examples of specific embodiments, to give a broad representation of the invention. Various changes and modifications within the spirit and scope of the invention will become apparent to those skilled in the art from this description and by practice of the invention. The scope of the invention is not intended to be limited to the particular forms disclosed and the invention covers all modifications, equivalents, and alternatives falling within the spirit and scope of the invention as defined by the claims.

Surface mass spectrometry techniques are limited in biological cell analyses, because most of the cell's contents are hidden in three-dimensional layers. Furthermore, ion suppression effects limit detection with the cells' complex matrix. The present invention provides an enabling technology for the analysis by the physical isolation of molecules of interest by chromatographic separation, increasing their concentration at the substrate surface, and reducing background providing detection levels of molecules in single cells that were not possible before. Further, substrates can be manufactured with specific physical characteristics to concentrate analytes, and molecules to promote the ionization of biological analytes, further improving the ability to analyze the contents of single cells.

A key enabling technology for the analysis of biological cells by surface mass spectrometers utilizes planar chromatography to improve detection levels of the contents of single cells. The present invention provides a system for preparing a biological cell for analysis. The system comprises the deposition of the biological cell on a planar chromatographic support medium and lysis of the biological cell. In one embodiment, the present invention includes one or two dimensional chromatography of the biological cell. One embodiment of the present invention provides a solvent to the planar chromatographic support medium to move the biological cell from its initial location. One embodiment of the present invention provides a planar chromatographic support medium including a planar unit and a porous material, a device for deposition of the biological cell on the planar chromatographic support medium, a device for cell lysis of the biological cell, and a chromatography device for one or two dimensional chromatography of the biological cell.

In various embodiments of the present invention, biological cells are prepared for analysis by: 1) deposition a on planar chromatographic support medium, followed by cell lysis and 1 or 2 dimensional chromatography, 2) or by growth on other rigid supports and contact transfer to a chromatographic support medium, followed by chromatography, or by 3) deposition, lysis and chromatography followed by contact transfer to an analysis medium. These methods accomplish cell lysis and chromatography on a disposable medium solving the problem of exposing biological cell contents to the surface analysis beam, concentrating analytes, reducing background interferences that prevent ionization. The fabrication and use of supports optimizing chromatographic separation and with chemical additives to improve chemical ionization are part of the present invention.

There are numerous uses for the present invention. The present invention is a key enabling technology for analysis of single cells by surface mass spectrometers such as ToF-SIMS, Nano-SIMS and MALDI mass spectrometers. Examples of specific uses the present invention are detecting markers for normal and cancerous cells, identifying of markers for chemical or radiation exposure damage, detection of genetic instability, and identifying cell origin. These methods will be used for medical diagnostic applications and for fundamental studies of environmental and medical conditions, involving individual eukaryotic and prokaryotic cells. The chromatographic supports for single cell analysis are expected to be a commercially viable product. Single cell planar chromatography can be the enabling technology for mass spectrometry-based medical diagnostics, and other environmental uses. Chromatographic media, optimized as to thickness and composition for biological cell analysis is expected to be a commercial product.

The invention is susceptible to modifications and alternative forms. Specific embodiments are shown by way of example. It is to be understood that the invention is not limited to the particular forms disclosed. The invention covers all modifications, equivalents, and alternatives falling within the spirit and scope of the invention as defined by the claims.

### BRIEF DESCRIPTION OF THE DRAWINGS

The accompanying drawings, which are incorporated into and constitute a part of the specification, illustrate specific embodiments of the invention and, together with the general description of the invention given above, and the detailed description of the specific embodiments, serve to explain the principles of the invention.

FIG. 1 is an illustration of a planar chromatographic support medium.

FIG. 2 is an illustration of the planar chromatographic support medium with mask sections.

FIG. 3 is an illustration of the planar chromatographic support medium with the mask sections removed.

FIG. 4 is a side view of the planar chromatographic support medium.

FIG. 5 is an illustration of an enlarged circular section the planar chromatographic support medium.

FIG. 6 illustrates another embodiment of an apparatus for preparing biological cell for analysis.

### DETAILED DESCRIPTION OF THE INVENTION

Referring to the drawings, to the following detailed description, and to incorporated materials, detailed information about the invention is provided including the description of specific embodiments. The detailed description serves to explain the principles of the invention. The invention is susceptible to modifications and alternative forms. The invention is not limited to the particular forms disclosed. The invention covers all modifications, equivalents, and alternatives falling within the spirit and scope of the invention as defined by the claims.

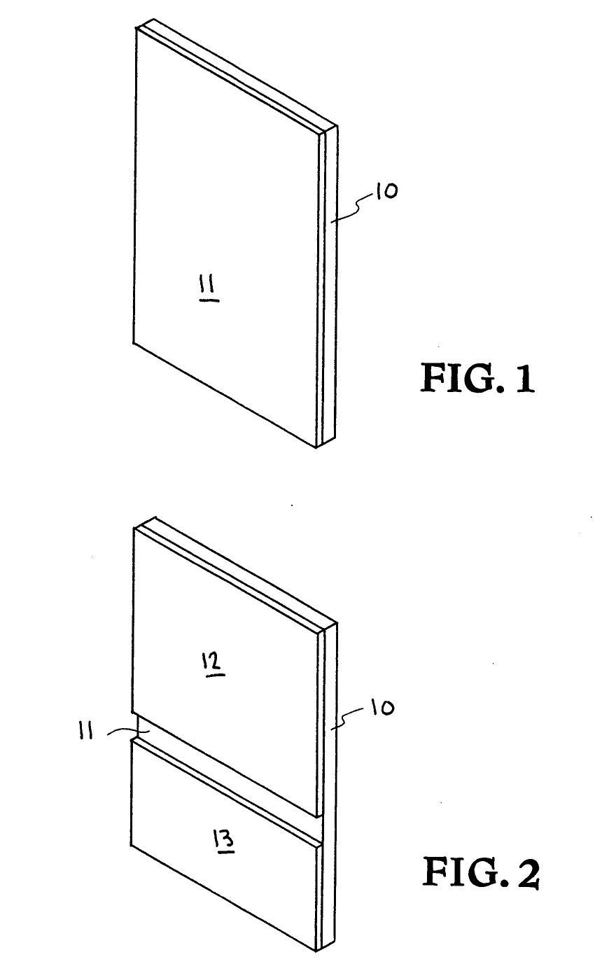

Referring now to FIGS. 1 through 5 of the drawings, an apparatus and method of preparing a biological cell for analysis by mass spectrometry of the present invention is illustrated. Surface mass spectrometry techniques are limited in biological cell analyses, because most of the cell's contents are hidden in three-dimensional layers. Furthermore, ion suppression effects limit detection with the cells' complex matrix. The present invention provides an enabling technology for the analysis by the physical isolation of molecules of interest by chromatographic separation, increasing their concentration at the substrate surface, and reducing background, providing detection levels of molecules in single cells that were not possible before. Further, substrates can be manufactured with specific physical characteristics to concentrate analyte molecules, and to promote the ionization of biological analytes, further improving the ability to analyze the contents of single cells.

Referring to FIG. 1, a planar chromatographic support medium is shown. The planar chromatographic support medium is designated by the reference numeral 10. The planar chromatographic support medium can be a silicon chip, a glass substrate, a circuit board material, or other material to facilitate instrumental analysis. A system for thin porous layers for thin-layer chromatography is described and illustrated in U.S. Pat. No. 6,395,178 issued to Heinz-Emil Hauck May 28, 2002. U.S. Pat. No. 6,395,178 issued to Heinz-Emil Hauck May 28, 2002 is incorporated herein by reference.

A porous coating material with specific physical characteristics to separate chemical components by chromatographic mechanisms is applied to the chromatographic support medium 10. As illustrated in FIG. 1, the planar chromatographic support medium 10 has a surface coating 11. The surface coating 11 is a porous coating material with specific physical characteristics to separate chemical components by chromatographic mechanisms.

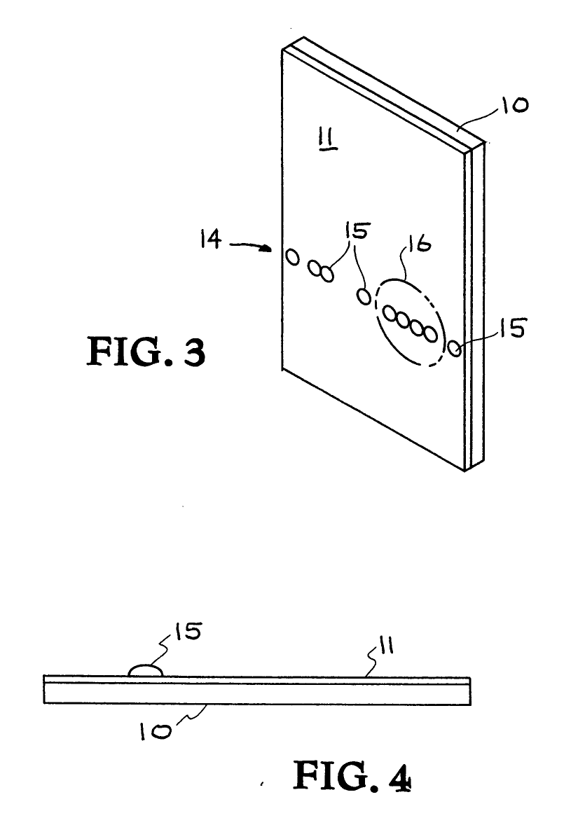

Referring now to FIG. 2, the planar chromatographic support medium 10 is shown with mask sections 12 and 13 applied to the planar chromatographic support medium 10. The mask sections 12 and 13 are applied to the surface of the planar chromatographic support medium 10 so as to leave a section the porous coating material surface 11 exposed. The cells under investigation are applied to the whole support medium. This can be accomplished by placing the support medium in a dish and seeding the whole dish with cells, allowing some to attach in the porous coating material 11. Alternatively, cells can be added on only a portion of the support medium. The mask sections 12 and 13 insure that the macroporous coating and the cells are located only on the section the surface 11 of the planar chromatographic support medium 10. The mask sections 12 and 13 are removed leaving the strip of cells 14 as shown in FIG. 3.

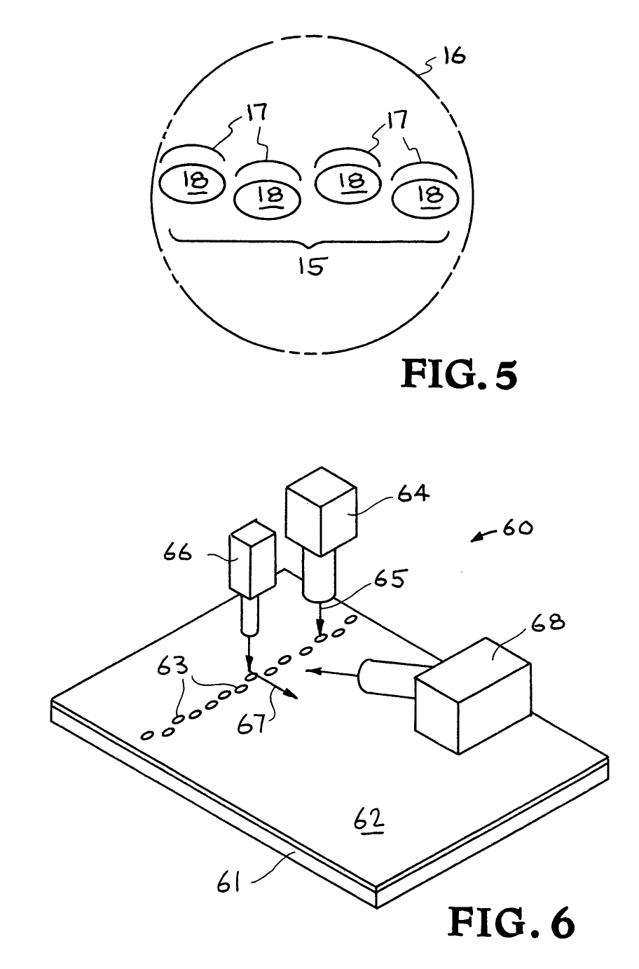

Referring now to FIG. 3, the planar chromatographic support medium 10 is shown with the mask sections 12 and 13 removed. The strip of cells 14 remains on the planar chromatographic support medium 10. The strip of cells 14 contains randomly placed individual cells 15. The cells 15 under investigation are lysed by heat, solvent dissolution, physical crushing or other means. A solvent is applied to the bottom edge of the support medium that travels through the support medium as a mobile phase to carry cell contents away from the original cell location. The circular section 16 designates an area of the strip of macroporous coating and cells 15 and cell products that is shown in greater detail in FIG. 5.

Referring now to FIG. 4, side view of the planar chromatographic support medium 10 is shown. The planar chromatographic support medium 10 has the coated macroporous surface 11. A cell 15 is shown on the macroporous coating surface 11 of the planar chromatographic support medium 10. For illustration purposes, a single cell 15 is shown in the macroporous coating 11. The planar chromatographic support medium 10 can be a silica chip or a material supporting a coating with specific physical characteristics to concentrate separate analyte molecules by one or more chromatographic mechanisms.

Referring now to FIG. 5, the circular section 16 is shown enlarged. The circular section 16 shows a portion of the row 14 of individual cells 15 on the macroporous coating 11 first illustrated in FIG. 3. The circular section 16 shows the cells 15 in greater detail. The cells under investigation have been lysed and contents separated by 1 dimensional chromatography. The cell contents 17 have been separated from the cell membranes 18.

The system preparing a biological cell for analysis by mass spectrometry of the present invention provides enabling technology for the analysis of biological cells by surface mass spectrometers. A system for the direct introduction of a sample taken from a planar electrophoresis into a mass spectrometer or other spectrometric device is described and illustrated in U.S. Pat. No. 5,208,458 issued to Kenneth L. Busch and Stephen M. Brown, Jr. May 4, 1993. U.S. Pat. No. 5,208,458 issued to Kenneth L. Busch and Stephen M. Brown, Jr. May 4, 1993 is incorporated herein by reference.

The present invention provides system wherein biological cells are prepared for analysis by: 1) deposition a on planar chromatographic support medium, followed by cell lysis and 1 or 2 dimensional chromatography, 2) or by growth on other rigid supports and contact transfer to a chromatographic support medium, followed by chromatography, or by 3) deposition, lysis and chromatography followed by contact transfer to an analysis medium. These methods accomplish cell lysis and chromatography on a disposable medium solving the problem of exposing biological cell contents to the surface analysis beam, concentrating analytes, reducing background interferences that prevent ionization. The fabrication and use of supports optimizing chromatographic separation and with chemical additives to improve chemical ionization are part of the present invention.

Referring to FIG. 6, another embodiment of an apparatus for preparing biological cell for analysis is illustrated. This embodiment is designated generally by the reference numeral 60. The apparatus 60 comprises a planar chromatographic support medium including a planar unit 61 and a porous material 62. A device deposits the biological cells on a limited section 63 of the planar chromatographic support medium. A device 64 for cell lysis of the biological cell provides lysis 65 of the biological cell. A chromatography device provides one or two dimensional chromatography of the biological cell.

The planar unit 61 can be a silicon chip, a glass substrate, a circuit board material, or other material to facilitate instrumental analysis. The porous material 62 can be a porous coating material with specific physical characteristics to separate chemical components by chromatographic mechanisms. The device 64 for cell lysis of the biological cell can be a device for heating, solvent dissolution, or physical crushing the biological cell. A device 66 applies a solvent near the bottom edge of the planar chromatographic support medium. The solvent travels through the support medium as a mobile phase to carry cell contents away from the original cell location as illustrated by the dotted arrow.

A device 68 provides analysis of the biological cell by surface mass spectrometers such as ToF-SIMS, Nano-SIMS and MALDI mass spectrometers. Systems for provides analysis of the biological cell by surface mass spectrometers are described and illustrated in U.S. Pat. No. 5,208,458 to Kenneth L. Busch and Stephen M. Brown, Jr. issued May 4, 1993. U.S. Pat. No. 5,208,458 issued to Kenneth L. Busch and Stephen M. Brown, Jr. May 4, 1993 is incorporated herein by reference.

While the invention may be susceptible to various modifications and alternative forms, specific embodiments have been shown by way of example in the drawings and have been described in detail herein. However, it should be understood that the invention is not intended to be limited to the particular forms disclosed. Rather, the invention is to cover all modifications, equivalents, and alternatives falling within the spirit and scope of the invention as defined by the following appended claims.

The invention claimed is:

1. A method of preparing a biological cell for analysis, comprising the step of:
deposition of the biological cell on a planar chromatographic support medium, and
lysis of the biological cell.

2. The method of preparing a biological cell for analysis of claim 1 wherein said step of deposition of the biological cell on planar chromatographic support medium comprises masking a portion of said planar chromatographic support medium leaving an un-masked area and depositing said biological cell on said un-masked area.

3. The method of preparing a biological cell for analysis of claim 1 wherein said step of deposition of the biological cell on planar chromatographic support medium comprises growth of the biological cell on a rigid support and contact transfer of the biological cell to a chromatographic support medium.

4. The method of preparing a biological cell for analysis of claim 1 wherein said step of lysis of the biological cell comprises heating the biological cell.

5. The method of preparing a biological cell for analysis of claim 1 wherein said step of lysis of the biological cell comprises solvent dissolution the biological cell.

6. The method of preparing a biological cell for analysis of claim 1 wherein said step of lysis of the biological cell comprises crushing the biological cell.

7. The method of preparing a biological cell for analysis of claim 1 wherein said step of deposition of the biological cell on a planar chromatographic support medium locates the biological cell at a first location on said planar chromatographic support medium and including the step of applying a solvent to said planar chromatographic support medium moves the biological cell from said first location.

8. The method of preparing a biological cell for analysis of claim 1 including one or two dimensional chromatography of the biological cell.

9. A method of preparing a biological cell for analysis, comprising the step of:
deposition of the biological cell on planar chromatographic support medium, and
one or two dimensional chromatography of the biological cell.

10. The method of preparing a biological cell for analysis of claim 9 wherein said step of deposition of the biological cell on planar chromatographic support medium comprises masking a portion of said planar chromatographic support medium leaving an un-masked area and depositing said biological cell on said un-masked area.

11. The method of preparing a biological cell for analysis of claim 9 wherein said step of deposition of the biological cell on planar chromatographic support medium comprises growth of the biological cell on a rigid support and contact transfer of the biological cell to a chromatographic support medium.

12. The method of preparing a biological cell for analysis of claim 9 wherein said step of deposition of the biological cell on a planar chromatographic support medium locates the biological cell at a first location on said planar chromatographic support medium and including the step of applying a solvent to said planar chromatographic support medium moves the biological cell from said first location.

13. A method of preparing a biological cell for analysis of claim 9 including cell lysis of the biological cell.

14. The method of preparing a biological cell for analysis of claim 13 wherein said step of lysis of the biological cell comprises heating the biological cell.

15. The method of preparing a biological cell for analysis of claim 13 wherein said step of lysis of the biological cell comprises solvent dissolution the biological cell.

16. The method of preparing a biological cell for analysis of claim 13 wherein said step of lysis of the biological cell comprises crushing the biological cell.

17. An apparatus for preparing biological cell for analysis, comprising:
a planar chromatographic support medium including a planar unit and a porous material,
a device for deposition of the biological cell on said planar chromatographic support medium,
a device for cell lysis of the biological cell, and
a chromatography device for one or two dimensional chromatography of the biological cell.

18. The apparatus for preparing biological cell for analysis of claim 17 wherein said planar unit is a silicon chip.

19. The apparatus for preparing biological cell for analysis of claim 17 wherein said device for cell lysis of the biological cell is a device for heating said biological cell.

20. The apparatus for preparing biological cell for analysis of claim 17 wherein said device for deposition of the biological cell on said planar chromatographic support medium locates the biological cell at a first location on said planar chromatographic support medium and including a device for applying solvent to the planar chromatographic support medium causing the biological cell to travel away from said first location.
